(19) United States
(12) Patent Application Publication
(10) Pub. No.: US 2005/0008605 A1
(43) Pub. Date: Jan. 13, 2005

(54) Composition containing water-soluble polymer

(75) Inventor: Florence L'Alloret, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/876,570

(22) Filed: Jun. 28, 2004

Related U.S. Application Data
(60) Provisional application No. 60/487,246, filed on Jul. 16, 2003.

(30) Foreign Application Priority Data
Jul. 2, 2003 (FR) 0308026

(51) Int. Cl. A61K 7/06; A61K 7/11

(52) U.S. Cl. 424/70.16

(57) ABSTRACT

The present invention relates to an aqueous composition containing at least one water-soluble polymer having an AMPS-based water-soluble skeleton and side chains comprising at least one polyoxyethylene block and at least one of a polyoxypropylene and polyoxybutylene block.

### REFERENCE TO PRIOR APPLICATIONS

This application claims priority to U.S. provisional application 60/487,246 filed Jul. 16, 2003, and to French patent application 0308026 filed Jul. 2, 2003, both incorporated herein by reference.

### FIELD OF THE INVENTION

The present invention relates to an aqueous composition, preferably suitable for topical application, containing at least one water-soluble polymer. Preferably the water soluble polymer has an AMPS-based water-soluble skeleton and side chains comprising at least one polyoxyethylene block and at least one polyoxypropylene and/or polyoxybutylene block.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The majority of cosmetic compositions contain one or more thickeners or gelling agents used to adjust the texture properties as a function of the intended application. The galenical supports most commonly used in cosmetics are, on the one hand, aqueous lotions or sera not comprising a fatty phase, and, on the other hand, emulsions, which are dispersions of two immiscible liquids such as water and oil. Cosmetic compositions may also comprise solid particles such as pigments, mineral UV-screening agents or fillers.

The main hydrophilic thickeners or gelling agents used are crosslinked polymeric gelling agents and natural polymers. Examples of crosslinked polymeric gelling agents that may be mentioned include carboxyvinyl polymers, for instance the products sold under the names Carbopol (INCI name: carbomer) by the company Noveon, polyacrylamides, polymers derived from 2-acrylamido-2-methylpropanesulfonic acid (AMPS), such as the product sold by the company Clariant under the name Hostacerin AMPS, and crosslinked anionic copolymers of acrylamide and of AMPS, such as the product sold under the name Sepigel 305 by the company SEPPIC. Examples of natural polymers that may be mentioned include xanthan gum and guar gum, or alternatively cellulose derivatives, starches and alginates. These natural polymers are generally used in combination with the crosslinked polymeric gelling agents, since their thickening power is usually not great enough for them to be able to be used alone.

These compounds undergo a reduction in gelling power when the temperature increases, which is reflected by fluidization of the formulations at skin temperature (about 32° C.). Formulations that are relatively fluid at ambient temperature are, as a result, difficult to apply to the face, and compositions that are more gelled all become more fluid when applied to the skin.

Moreover, the stability of cosmetic compositions as a function of time and over a wide temperature range (5° C.-50° C.) is a crucial parameter to consider during the development of a new composition. The presence of a liquid dispersed phase (in emulsions) or solid dispersed phase (in suspensions) can cause destabilization phenomena such as sedimentation, creaming or aggregation and coalescence, this destabilization possibly being reflected at the microscopic and/or macroscopic level. One solution for limiting these destabilization phenomena consists in increasing the viscosity of the continuous phase, for example using the thickeners/gelling agents mentioned previously in the case of an aqueous continuous phase, which involves a high concentration of polymer in the composition, which then has a sticky, film-forming feel after application to the skin.

### SUMMARY OF THE INVENTION/DISCUSSION OF CONVENTIONAL ART

It is thus seen that it is necessary to provide novel hydrophilic gelling agents for diversifying the range of textures of aqueous cosmetic compositions, especially with the possibility of transforming a composition during application to the skin, from a fluid state to a gelled state, and also of improving the stability of compositions beyond 30° C., specifically for compositions that are sparingly gelled at room temperature.

Particular polymers whose solubility in water is modified beyond a certain temperature are known in the prior art. These are polymers with a heat-demixing temperature (or cloud point), thus defining their zone of solubility in water. The minimum demixing temperature obtained as a function of the polymer concentration is known as the “LCST” (Lower Critical Solution Temperature).

Thus, documents WO-A-98/29487 and WO-A-97/00275 especially disclose a reversible gelling or thickening polymer system comprising a composition that is soluble in the medium under consideration, consisting of an associative compound capable of aggregating during an increase in temperature, and of a compound that is soluble in the medium under consideration and linked to the associative compound. However, the compounds illustrated in document WO-A-98/29487 have the drawback of being sensitive in the presence of multivalent ions and of being pH-sensitive. Furthermore, it is not easy to control the structure and chemical nature of the polymers described in documents WO-A-97/00275 and WO-A-98/29487.

Moreover, document EP-A-1 307 501 relates to polymers comprising water-soluble units and units with an LCST, whose heat-demixing temperature at 1% in water is between 5° C. and 40° C. The units with an LCST may be random copolymers of oxyethylene and of oxypropylene. However, these random copolymers have the drawback of having a large amplitude of the heat-gelling effect, which thus intervenes over a wide temperature range.

One aim of the present invention is to overcome the drawbacks of the prior art. The inventor has found, surprisingly, water-soluble polymers capable of overcoming the drawbacks of those described in the prior art. These polymers differ from those of the prior art at least by the fact that they have an AMPS-based water-soluble skeleton and side chains comprising at least one polyoxyethylene block and at least one polyoxypropylene and/or at least one polyoxybutylene block.

### DETAILED DESCRIPTION OF THE PREFERRED EMBODIMENTS

Polymers having (comprising) an AMPS-based water-soluble skeleton and side chains comprising at least one polyoxyethylene block and at least one polyoxypropylene and/or at least one polyoxybutylene block have the advantage of being insensitive in the presence of multivalent ions, of being pH-insensitive and of providing compositions that have good cosmetic properties. In addition, compared with polymers comprising random copolymers of oxyethylene and oxypropylene, they have the advantage of having an amplitude of the heat-gelling effect that intervenes over a narrow temperature range, thus allowing a sharper fluid/gel transition on application to the skin and thus greater gelation of the composition applied to the skin.

The inventor has found that the distribution of the oxyethylene and oxypropylene/oxybutylene units in blocks was reflected by the existence of a critical temperature, known as the aggregation temperature, at the microscopic level, i.e. invisible to the naked eye, in contrast with random copolymers, which have, in water, a lower critical solution temperature, beyond which macroscopic separation of the phases is observed (cloud point visible to the naked eye). The phenomenon of aggregation of the block polymers at the microscopic level corresponds to a micellization for the polyoxyethylene/polyoxypropylene/polyoxyethylene triblock polymers, as described by Wanka et al. (Colloid and Polymer Science, 1990, (268), 101-117) and Linse et al. (Macromolecules, 1992, (25), 5434-5439), and the aggregation temperature at the microscopic level may be determined by microcalorimetry measurements, as described by Wanka et al. (Colloid and Polymer Science, 1990, (268), 101-117).

One preferred embodiment of the present invention is thus a composition suitable for topical application, especially a cosmetic or dermatological composition, comprising a physiologically acceptable medium and at least one water-soluble polymer having a water-soluble skeleton based on 2-acrylamido-2-methylpropanesulfonic acid and side chains comprising at least one polyoxyethylene block and at least one polyoxypropylene block and/or one at least one polyoxybutylene block.

In a preferred embodiiment the composition according to the invention is intended for topical application especially to the skin, the hair or the integuments, and contains a physiologically acceptable medium. The term “physiologically acceptable medium” means a medium that is compatible with keratin materials and especially the skin, the lips, the nails, the scalp and/or the hair.

The polymers used in the composition of the invention preferably are grafted polymers, although the manner in which the polymers are prepared is not limited, and have a molecular architecture where the water-soluble skeleton bears side chains comprising at least one polyoxyethylene block and at least one polyoxypropylene and/or polyoxybutylene block. Thus, the side chains comprise, at a minimum, one polyoxyethylene block and one block of polyoxypropylene or polyoxybutylene. However, the invention polymers also include side chains with multiple blocks of polyoxyethylene, polyoxypropylene and polyoxybutylene as long as the minimum requirement is met. In this regard the invention water-soluble polymer may be described as comprising a water-soluble 2-acrylamido-2-methyl-propanesulfonic acid skeleton and at least one side chain, said side chain comprising 1) at least one polyoxyethylene block and 2) at least one block selected from the group consisting of a polyoxypropylene block and a polyoxybutylene block. These structures may be partially crosslinked, for example with a degree of crosslinking of from 0.01 mol % to 10 mol % as indicated below.

These polymers may be obtained from:

(A) 70 mol % to 99.5 mol % of 2-acrylamido-2-methylpropanesulfonic acid (AMPS) units of formula (I): 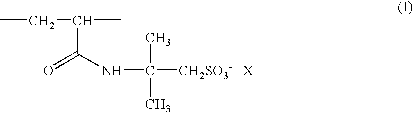
in which X⁺ is a proton, an alkali metal cation, an alkaline-earth metal cation or an ammonium ion;

(B) 0.5 mol % to 30 mol % and especially from 2 mol % to 25 mol % of units of formula (II) below: 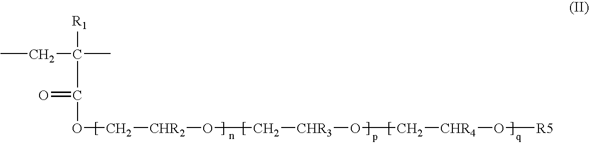
in which:
n, p and q, independently of each other, denote an integer and range from 0 to 200;
R₁ denotes a hydrogen atom or a linear or branched alkyl radical containing from 1 to 6 carbon atoms;
R₂, R₃ and R₄ denote a hydrogen atom or an alkyl radical containing 1 or 2 carbon atoms, at least one of the radicals R₂, R₃ or R₄ being a hydrogen atom, and at least one of the radicals R₂, R₃ or R₄ being an alkyl radical containing one or two carbon atoms;
R₅ denotes a hydrogen atom or a linear, cyclic or branched alkyl radical containing from 1 to 18 carbon atoms;

(C) 0 to 30 mol % of water-soluble or hydrophobic units bearing, before polymerization, at least one unsaturated function.

Preferably, the microscopic-level aggregation temperature of the polyethers of block structure of formula (II), at 1% in water, ranges from 5° C. to 40° C. and more specifically from 10° C. to 30° C.

Preferably, the unit of formula (II) is chosen from those in which:
one of the radicals R₂ or R₃ is a hydrogen atom and the other is an alkyl radical containing one or two carbon atoms, and the integer q is equal to 0, n and p being integers between 0 and 200. As commercially available copolymers corresponding to this formula, examples that may be mentioned include the polyoxyethylene/polyoxypropylene alkyl ether diblock copolymers sold by the company NOF under the references 502134 (R₅ is a butyl group), 523216 (R₅ is a lauryl group), 105428, 500442, 109884 (R₅ is a cetyl group), 521034 and 522074 (R₅ is a stearyl group); or alternatively
R₂ is a hydrogen atom, R₃ is a methyl radical and R₄ and R₅ are hydrogen atoms, n, p and q being integers between 0 and 200. Examples of commercially available copolymers corresponding to this formula that may be mentioned include the polyoxyethylene/polyoxypropylene/polyoxyethylene triblock copolymers sold by the company BASF under the name Pluronic, and especially Pluronic F-127 (n=106, p=69 and q=106), Pluronic P-123 (n=20, p=70 and q=20), Pluronic P-103 (n=18, p=58 and q=18), Pluronic P-104 (n=25, p=58 and q=25), Pluronic P-105 (n=38, p=58 and q=38), Pluronic L-121 (n=5, p=69 and q=5) and Pluronic L-122 (n=10, p=69 and q=10); or alternatively
R₂ is a hydrogen atom, R₃ is an ethyl radical and R₄ and R₅ are hydrogen atoms. Examples of commercially available copolymers corresponding to this formula that may be mentioned include the polyoxyethylene/polyoxybutylene/polyoxyethylene triblock copolymer sold by the company NOF under the name Plonon B208 (n=80, p=28 and q=80).

The “water-soluble unit” that may be used as compound (C) means units that are soluble in water, from 5° C. to 80° C., in a proportion of at least 10 g/l and preferably at least 20 g/l.

The hydrophobic units, when they are present as compound (C), must be present in an amount that is small enough for them not to adversely affect the solubility of the skeleton of the polymer in water.

The water-soluble units are obtained from one or more water-soluble monomers. As water-soluble monomers that may be used as precursors of the water-soluble units, alone or as a mixture, examples that may be mentioned include the following monomers, which may be in acid form and in salt form:
(meth)acrylic acid;
styrenesulfonic acid;
vinylsulfonic acid and (meth)allylsulfonic acid;
(meth)acrylamide;
vinylphosphonic acid;
N-vinylacetamide and N-methyl-N-vinylacetamide;
N-vinylformamide and N-methyl-N-vinylformamide;
N-vinyllactams comprising a cyclic alkyl group of 4 to 9 carbon atoms, such as N-vinylpyrrolidone, N-butyrolactam or N-vinylcaprolactam;
maleic anhydride;
itaconic acid;
vinyl alcohol of formula CH₂═CHOH;
vinyl ethers of formula CH₂═CHOR in which R is a linear or branched, saturated or unsaturated hydrocarbon-based radical containing from 1 to 6 carbons;
dimethyldiallylammonium chloride;
quaternized dimethylaminoethyl methacrylate (DMAEMA);
(meth)acrylamidopropyltrimethylammonium chloride (APTAC and MAPTAC);
methylvinylimidazolium chloride;
2-vinylpyridine and 4-vinylpyridine;
acrylonitrile;
glycidyl (meth)acrylate;
vinyl chloride and vinylidene chloride;
vinyl monomers of formula (A) below: 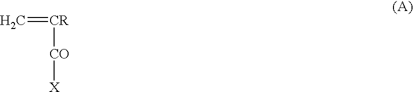
   in which:
R is chosen from H, —CH₃, —C₂H₅ and —C₃H₇;
X is chosen from:
   alkyl oxides of —OR′ type in which R′ is a linear or branched, saturated or unsaturated hydrocarbon-based radical containing from 1 to 6 carbon atoms, optionally substituted with at least one halogen atom (iodine, bromine, chlorine or fluorine); a sulfonic (—SO₃⁻), sulfate (—SO₄⁻), phosphate (—PO₄H₂); hydroxyl (—OH); primary amine (—NH₂); secondary amine (—NHR₆), tertiary amine (—NR₆R₇) or quaternary amine (—N⁺R₆R₇R₈) group with R₆, R₇ and R₈ being, independently of each other, a linear or branched, saturated or unsaturated hydrocarbon-based radical containing 1 to 6 carbon atoms, with the proviso that the sum of the carbon atoms of R′+R₆+R₇+R₈ does not exceed 6;
   —NH₂, —NHR′ and —NR′R″ groups in which R′ and R″ are, independently of each other, linear or branched, saturated or unsaturated hydrocarbon-based radicals containing 1 to 6 carbon atoms, with the proviso that the total number of carbon atoms of R′+R″ does not exceed 6, the said R′ and R″ optionally being substituted with a halogen atom (iodine, bromine, chlorine or fluorine); a hydroxyl (—OH); sulfonic (—SO₃⁻); sulfate (—SO₄⁻); phosphate (—PO₄H₂); primary amine (—NH₂); secondary amine (—NHR₆), tertiary amine (—NR₆R₇) and/or quaternary amine (—N⁺R₆R₇R₈) group with R₆, R₇ and R₈ being, independently of each other, a linear or branched, saturated or unsaturated hydrocarbon-based radical containing 1 to 6 carbon atoms, with the proviso that the sum of the carbon atoms of R′+R″+R₆+R₇+R₈ does not exceed 6. Examples of compounds corresponding to this formula that may be mentioned include N,N-dimethylacrylamide and N,N-diethylacrylamide;
and mixtures thereof.

The hydrophobic units are obtained from one or more hydrophobic monomers. As hydrophobic monomers that may be used as precursors of the hydrophobic units, examples that may be mentioned include the following monomers, alone or as a mixture:
styrene and its derivatives, such as 4-butylstyrene, α-methylstyrene and vinyltoluene,
vinyl acetate of formula CH₂═CH—OCOCH₃;
vinyl ethers of formula CH₂═CHOR in which R is a linear or branched, saturated or unsaturated hydrocarbon-based radical containing from 1 to 6 carbon atoms;
acrylonitrile,
caprolactone,
vinyl chloride and vinylidene chloride,
silicone derivatives, leading, after polymerization, to silicone polymers such as methacryloxypropyltris(trimethylsiloxy)silane and silicone methacrylamides,
the hydrophobic vinyl monomers of formula (B) below: 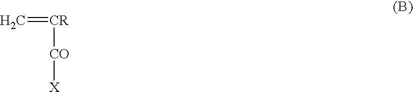
   in which:
R is chosen from H, —CH₃, —C₂H₅ and —C₃H₇,
X is chosen from:
   alkyl ethers of the type —OR′ in which R′ is a linear or branched, saturated or unsaturated hydrocarbon-based radical containing from 1 to 6 carbon atoms. Examples of compounds corresponding to this formula that may be mentioned include methyl methacrylate, ethyl methacrylate, n-butyl (meth)acrylate, tert-butyl (meth)acrylate, cyclohexyl acrylate, isobornyl acrylate and 2-ethylhexyl acrylate;
   groups —NH₂, —NHR′ and —NR′R″ in which R′ and R″ are, independently of each other, linear or branched, saturated or unsaturated hydrocarbon-based radicals containing from 1 to 6 carbon atoms, with the proviso that the total number of carbon atoms in R′+R″ does not exceed 6,
and mixtures thereof.

The ionizable water-soluble units, i.e., especially, the units C and/or the AMPS chain constituting the water-soluble skeleton, are preferably partially or totally neutralized with a mineral base (for example sodium, ammonium, lithium, calcium, magnesium or ammonium substituted with 1 to 4 alkyl groups containing from 1 to 15 carbon atoms) or an organic base such as monoethanolamine, diethanolamine, triethanolamine, aminomethylpropanediol, N-methylglucamine and basic amino acids, for instance arginine and lysine, and mixtures thereof.

The water-soluble skeleton of the polymer used according to the invention may be crosslinked using polyolefinically unsaturated compounds commonly used as crosslinking agents for the crosslinking of polymers obtained by free-radical polymerization. Examples of crosslinking agents that may be mentioned include divinylbenzene, diallyl ether, triallylamine, tetraallylethylenediamine, dipropylene glycol diallyl ether, polyglycol diallyl ethers, hydroquinone diallyl ether, trimethylolpropane diallyl ether, tetraallyl-oxyethane, allylic ethers of alcohols of the sugar series, polyallyl esters, tetraallyloxyethanoyl or other polyfunctional alcohol allyl or vinyl ethers, triethylene glycol divinyl ether, allylic esters of vinylphosphonic acid and of phosphoric acid, compounds comprising two or three (meth)acrylate or (meth)acrylamide groups, such as ethylene glycol diacrylate, tetraethylene glycol diacrylate, butanediol diacrylate, allyl methacrylate, trimethylolpropane triacrylate (TMPTA) and methylenebisacrylamide, or mixtures thereof.

According to one preferred embodiment of the invention, the crosslinking agent is preferably chosen from methylenebisacrylamide, allyl methacrylate and trimethylolpropane triacrylate (TMPTA). The degree of crosslinking preferably ranges from 0.01 mol % to 10 mol % and more particularly from 0.2 mol % to 7 mol % relative to the polymer.

Preferably, the water-soluble skeleton has a molar mass of between 1000 g/mol and 50 000 000 g/mol and preferably between 10 000 g/mol and 10 000 000 g/mol.

The polymers used according to the invention may be obtained via the synthetic processes conventionally used for obtaining “comb” polymers. Examples that may be mentioned include the following synthetic routes:
Reaction for grafting polyether chains containing at least one reactive end onto water-soluble macromolecules bearing complementary reactive sites, for instance the reaction of a polyether containing an amino end with a water-soluble polymer bearing carboxylic acid functions, in the presence of a coupling agent such as dicyclohexylcarbodiimide or 1-(3-dimethylaminopropyl)-3-ethylcarbodiimide hydrochloride. The polymers obtained have a “comb” structure. This synthetic route is described, for example, by Hourdet (Hourdet D., L'Alloret F., Audebert R., Polymer, 1997, 38 (10), 2535-2547), and in documents EP-A-583 814, EP-A-629 649, WO-A-95/24430 and WO-A-02/09064.
Copolymerization of a polyether macromonomer and at least one water-soluble monomer. This synthetic route in homogeneous medium is described by Hourdet (Hourdet D., L'Alloret F., Audebert R., Polymer, 1997, 38 (10), 2535-2547), and in documents EP-A-583 814, EP-A-629 649, WO-A-95/24430 and WO-A-02/09064. The polyether macromonomer may be obtained by reaction between a polyether bearing at least one reactive site and a compound bearing at least one complementary reactive site and at least one unsaturated hydrocarbon-based bond such as a vinyl or allyl function. Examples of compounds of this type that may be mentioned include (meth)acrylic acid, maleic anhydride, itaconic acid, vinyl alcohol, vinyl acetate, glycidyl (meth)acrylate, 3-chloropropene, 4-isocyanatostyrene and chloromethylstyrene. A reactive site may be chosen especially from alcohol, glyceryl ester, isocyanate, primary, secondary or tertiary amine, carboxylic acid and halogen functions. In particular, a reactive site of the carboxylic acid or ester type will generally react with a reactive site of the alcohol or amine type; an isocyanate site will rather react with an alcohol site, and a halogen site with an alcohol or amine site. The reactions involved may be, for example, an esterification, a transesterification, an amidation or a nucleophilic substitution.

The polymerization process for preparing the polymers used in the composition according to the invention may be performed as an inverse emulsion, as described in document WO-A-00/35961. This synthetic process as described in documents EP-A-1 069 142 and EP-A-1 260 531 is performed in an aqueous-alcoholic medium, leading to a free-radical precipitation polymerization. The alcohol used is water-miscible and contains from 1 to 4 carbon atoms, for instance methanol, ethanol, propanol, isopropanol and, preferably, tert-butanol. The monomer(s) and the macromonomer are totally or partially dissolved in the polymerization medium, whereas the polymer is insoluble therein. The polymerization reaction is performed at a temperature of between −10° C. and 100° C. and preferably between 20° C. and 70° C. The polymerization is performed in inert medium, preferably under a nitrogen or argon atmosphere. The polymer appears in the reaction medium in the form of a white precipitate. It may be readily isolated using the usual separation, evaporation and drying processes. For example, the alcohol such as tert-butanol may be removed by filtration or distillation.

The polymers used in the composition according to the invention are preferably present in an amount preferably ranging from 0.01% to 20% by weight, especially from 0.05% to 15% by weight, in particular from 0.1% to 15% by weight and better still from 0.1% to 10% by weight relative to the total weight of the composition.

It is possible to add to the said composition according to the invention the constituents usually used in the intended type of application. Needless to say, a person skilled in the art will take care to select these optional additional constituents, and/or the amount thereof, such that the advantageous properties of the composition according to the invention are not, or are not substantially, adversely affected by the envisaged addition.

The composition according to the invention is an aqueous composition. It thus contains water, constituting an aqueous phase in which is found the polymer used according to the invention. The water is preferably present in an amount preferably ranging from 20% to 99.9% by weight, especially from 40% to 95% by weight, relative to the total weight of the composition.

The composition may also contain one or more water-soluble and/or water-dispersible compounds, for instance polyols such as dipropylene glycol, glycerol and butylene glycol, surfactants, water-soluble or water-dispersible organic and mineral UV-screening agents, active agents, salts, fillers, organic particles and hydrophilic polymers. Examples of hydrophilic polymers that may be mentioned include modified or unmodified carboxyvinyl polymers, such as the products sold under the names Carbopol (INCI name: carbomer) and Pemulen (INCI name: acrylates/C10-30 alkyl acrylate crosspolymer) by the company Noveon; polyacrylamides; optionally crosslinked and/or neutralized 2-acrylamido-2-methylpropanesulfonic acid polymers and copolymers, for instance the poly(2-acrylamido-2-methylpropanesulfonic acid) sold by the company Clariant under the name “Hostacerin AMPS” (INCI name: ammonium polyacryldimethyltauramide); crosslinked anionic copolymers of acrylamide and of AMPS, which are in the form of a W/O emulsion, such as those sold under the name Sepigel 305 (CTFA name: polyacrylamide/C13-14 isoparaffin/laureth-7) and under the name Simulgel 600 (CTFA name: acrylamide/sodium acryloyldimethyltaurate copolymer/isohexadecane/polysorbate 80) by the company SEPPIC; synthetic neutral polymers such as poly-N-vinylpyrrolidone; polysaccharides, for instance guar gum, xanthan gum and cellulose derivatives, water-soluble or water-dispersible silicone derivatives, for instance acrylic silicones, polyether silicones and cationic silicones.

The composition of the invention may also comprise at least one fatty phase, also referred to as the oily phase, and may thus constitute water-in-oil (W/O) or oil-in-water (O/W) or multiple (W/O/W or O/W/O) emulsions, and preferably oil-in-water (O/W) or multiple (W/O/W) emulsions. When it is present, the oily phase may represent, for example, from 0.5% to 80% by weight, for example from 1% to 80% by weight, better still from 2% to 70% by weight, even better from 5% to 60% by weight and preferably from 10% to 40% by weight relative to the total weight of the composition.

The oily phase contains at least one oil, more particularly at least one cosmetic oil. The term “oil” means a fatty substance that is liquid at room temperature (25° C.).

As oils that may be used in the composition of the invention, examples that may be mentioned include:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, coriander oil, castor oil, avocado oil, caprylic/capric acid triglycerides, for instance those sold by the company Stearineries Dubois or those sold under the names Miglyol 810, 812 and 818 by the company Dynamit Nobel, jojoba oil or shea butter oil;
synthetic esters and synthetic ethers, especially of fatty acids, for instance oils of formulae R¹COOR² and R¹OR² in which R¹ represents a fatty acid residue containing from 8 to 29 carbon atoms, and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, isohexadecane, isododecane, and hydrogenated polyisobutene such as Parleam® oil;
partially hydrocarbon-based and/or silicone-based fluoro oils, for instance those described in document JP-A-2 295 912;
silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones), for instance cyclohexadimethylsiloxane and cyclopentadimethylsiloxane polydimethylsiloxanes comprising alkyl, alkoxy or phenyl groups, that are pendent or at the end of a silicone chain, these groups containing from 2 to 24 carbon atoms; phenylsilicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyltrimethyl siloxysilicates and polymethylphenylsiloxanes, and linear or branched, non-cyclic volatile silicone oils;
mixtures thereof.

The oily phase may comprise other fatty substances, for instance fatty acids containing from 8 to 30 carbon atoms, for instance stearic acid, lauric acid, palmitic acid and oleic acid; waxes, for instance lanolin, beeswax, carnauba wax or candelilla wax, paraffin wax, lignite wax or microcrystalline waxes, ceresin or ozokerite, and synthetic waxes, for instance polyethylene waxes and Fischer-Tropsch waxes; gums such as silicone gums (dimethiconol); silicone resins such as trifluoromethyl-C1-4-alkyldimethicone and trifluoropropyldimethicone; silicone elastomers, for instance the products sold under the name “KSG” by the company Shin-Etsu, under the names “Trefil”, “BY29” and “EPSX” by the company Dow Corning, or under the name “Gransil” by the company Grant Industries. These fatty substances may be chosen in a varied manner by a person skilled in the art so as to prepare a composition having the desired properties, for example in terms of consistency or texture.

When the composition is an emulsion, it may also contain an emulsifying surfactant.

As examples of emulsifying surfactants for the W/O emulsions, mention may be made, for example, of dimethicone copolyols such as the mixture of cyclomethicone and of dimethicone copolyol, sold under the name DC 5225 C by the company Dow Corning or the dimethicone copolyol sold under the name Abil EM 97 by the company Goldschmidt, and alkyldimethicone copolyols such as the laurylmethicone copolyol sold under the name 5200 Formulation Aid by the company Dow Corning, the cetyldimethicone copolyol sold under the name Abil EM 90 by the company Goldschmidt, or the polyglyceryl-4 isostearate/cetyldimethicone copolyol/hexyl laurate mixture sold under the name Abil WE 09 by the company Goldschmidt. One or more co-emulsifiers may also be added thereto. Advantageously, the co-emulsifier may be chosen from the group comprising polyol alkyl esters. Polyol alkyl esters that may especially be mentioned include glycerol and/or sorbitan esters, for example polyglyceryl isostearate such as the product sold under the name Isolan GI 34 by the company Goldschmidt, sorbitan isostearate, such as the product sold under the name Arlacel 987 by the company ICI, glyceryl sorbitan isostearate, such as the product sold under the name Arlacel 986 by the company ICI, and mixtures thereof.

For the O/W emulsions, examples of emulsifiers that may be mentioned include nonionic surfactants, especially such as esters of polyols and of fatty acid with a saturated or unsaturated chain containing, for example, from 8 to 24 carbon atoms and better still from 12 to 22 carbon atoms, and the oxyalkylenated derivatives thereof, i.e. derivatives comprising oxyethylene and/or oxypropylene units, for instance glyceryl esters of C₈-C₂₄ fatty acids, and the oxyalkylenated derivatives thereof; polyethylene glycol esters of C₈-C₂₄ fatty acids, and the oxyalkylenated derivatives thereof; sorbitol esters of C₈-C₂₄ fatty acids, and the oxyalkylenated derivatives thereof; sugar (sucrose, glucose or alkylglucose) esters of C₈-C₂₄ fatty acids, and the oxyalkylenated derivatives thereof; fatty alcohol ethers; sugar ethers of C₈-C₂₄ fatty alcohols, and mixtures thereof.

The composition may also contain any active agent that is suitable for the desired aim. Active agents that may especially be mentioned include vitamins (A, C, E, K, PP, etc.), alone or as a mixture, and also derivatives thereof; keratolytic and/or desquamating agents such as salicylic acid and its derivatives, α-hydroxy acids, ascorbic acid and its derivatives, anti-inflammatory agents, calmatives, depigmenting agents, tensioning agents such as synthetic polymers, plant proteins, polysaccharides of plant origin, which may or may not be in the form of microgels, starches, wax dispersions, mixed silicates and colloidal particles of mineral fillers; matting agents, agents for preventing hair loss and/or for promoting regrowth of the hair, or alternatively antiwrinkle agents, and mixtures thereof.

The composition according to the invention may contain one or more mineral or organic fillers. As mineral fillers that may be present in the composition according to the invention, examples that may be mentioned include mineral pigments, silica powder, talc and colloidal particles of mineral fillers such as colloidal silica nanoparticles, for instance the product sold under the name Cosmo S40 by the company Catalysts and Chemicals and the product sold under the name Ludox AM by the company Grace. Examples of organic fillers that may be mentioned include polyamide particles and especially those sold under the name Orgasol by the company Atochem; polyethylene powders; microspheres based on acrylic copolymers, such as those made of ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as maize starch, wheat starch or rice starch, which may or may not be crosslinked, such as the starch powders crosslinked with octenylsuccinic anhydride, sold under the name Dry-Flo by the company National Starch; silicone resin microbeads, such as those sold under the name Tospearl by the company Toshiba Silicone; and mixtures thereof. When they are present in the compositions according to the invention, the fillers are for example in proportions ranging from 0.05% to 30% by weight relative to the total weight of the composition, and preferably ranging from 0.5% to 20% by weight relative to the total weight of the composition.

The UV-screening agents or photoprotective agents that may be used in the composition in accordance with the invention may be chosen from organic photoprotective agents and/or at least the mineral photoprotective agents that are UVA-active and/or UVB-active (absorbers), which are water-soluble or liposoluble, or else insoluble in the cosmetic solvents commonly used.

The organic photoprotective agents are especially chosen from anthranilates; cinnamic derivatives; dibenzoylmethane derivatives; salicylic derivatives; camphor derivatives; triazine derivatives such as those described in documents U.S. Pat. No. 4,367,390, EP-A-863 145, EP-A-517 104, EP-A-570 838, EP-A-796 851, EP-A-775 698, EP-A-878 469, EP-A-933 376, EP-A-507 691, EP-A-507 692, EP-A-790 243 and EP-A-944 624; benzophenone derivatives; β,β-diphenylacrylate derivatives; benzotriazole derivatives; benzalmalonate derivatives; benzimidazole derivatives; imidazolines; bis-benzazolyl derivatives as described in documents EP-A-669 323 and U.S. Pat. No. 2,463,264; p-aminobenzoic acid (PABA) derivatives; methylenebis(hydroxyphenylbenzotriazole) derivatives as described in documents U.S. Pat. No. 5,237,071, U.S. Pat. No. 5,166,355, GB-A-2 303 549, DE-A-197 26 184 and EP-A-893 119; screening polymers and screening silicones such as those described especially in document WO-A-93/04665; dimers derived from α-alkylstyrene such as those described in document DE-A-198 55 649.

As examples of UV-A-active and/or UV-B-active photoprotective agents, mention may be made more particularly of the following, denoted hereinbelow under their INCI name:
para-aminobenzoic acid derivatives, including the following: PABA, Ethyl PABA, Ethyl dihydroxypropyl PABA, Ethylhexyl dimethyl PABA sold in particular under the name “Escalol 507” by ISP, Glyceryl PABA, PEG-25 PABA sold under the name “Uvinul P25” by BASF;
salicylic derivatives, including the following: Homosalate sold in particular under the name “Eusolex HMS” by Rona/EM Industries, Ethylhexyl salicylate sold under the name “Neo Heliopan OS” by Haarmann and Reimer, Dipropylene glycol salicylate sold under the name “Dipsal” by Scher, TEA salicylate sold under the name “Neo Heliopan TS” by Haarnann and Reimer;
dibenzoylmethane derivatives, including the following: Butyl methoxydibenzoylmethane sold in particular under the trade name “Parsol 1789” by Hoffmann LaRoche, and Isopropyldibenzoylmethane;
cinnamic derivatives, including the following: Ethylhexyl methoxycinnamate sold in particular under the trade name “Parsol MCX” by Hoffmann LaRoche, Isopropyl methoxycinnamate, Isoamyl methoxycinnamate sold in particular under the trade name “Neo Heliopan E 1000” by Haarmann and Reimer, Cinoxate, DEA methoxycinnamate, Diisopropyl methylcinnamate, Glyceryl ethylhexanoate dimethoxycinnamate;
β,β-diphenylacrylate derivatives, including the following: Octocrylene sold in particular under the trade name “Uvinul N539” by BASF, Etocrylene sold in particular under the trade name “Uvinul N35” by BASF;
benzophenone derivatives, including the following: Benzophenone-1 sold in particular under the trade name “Uvinul 400” by BASF, Benzophenone-2 sold in particular under the trade name “Uvinul D50” by BASF, Benzophenone-3 or Oxybenzone sold in particular under the trade name “Uvinul M40” by BASF, Benzophenone-4 sold in particular under the trade name “Uvinul MS40” by BASF, Benzophenone-5, Benzophenone-6 sold in particular under the trade name “Helisorb 11” by Norquay, Benzophenone-8 sold in particularly under the trade name “Spectra-Sorb UV-24” by American Cyanamid, Benzophenone-9 sold in particular under the trade name “Uvinul DS-49” by BASF, Benzophenone-12, and n-hexyl 2-(4-diethylamino-2-hydroxybenzoyl)benzoate;
benzylidenecamphor derivatives, including the following: 3-Benzylidenecamphor, 4-Methylbenzylidenecamphor sold in particular under the name “Eusolex 6300” by Merck, Benzylidenecamphorsulfonic acid, Camphor benzalkonium methosulfate, Terephthalylidenedicamphorsulfonic acid (“Mexoryl SX” by Chimex), Polyacrylamidomethylbenzylidenecamphor;
benzimidazole derivatives, including the following: Phenylbenzimidazolesulfonic acid sold in particular under the trade name “Eusolex 232” by Merck, Disodium phenyldibenzimidazoletetrasulfonate sold in particular under the trade name “Neo Heliopan AP” by Haarmann and Reimer;
triazine derivatives, including the following: Anisotriazine sold in particular under the trade name “Tinosorb S” by Ciba Specialty Chemicals, Ethylhexyltriazone sold in particular under the trade name “Uvinul T150” by BASF, Diethylhexylbutamidotriazone sold in particular under the trade name “Uvasorb HEB” by Sigma 3V, 2,4,6-Tris(diisobutyl 4′-aminobenzalmalonate)-s-triazine;
benzotriazole derivatives, including the following: Drometrizole trisiloxane sold under the name “Silatrizole” by Rhodia Chimie, Methylenebis(benzotriazolyl)tetramethylbutylphenol sold in particular in solid form under the trade name “MIXXIM BB/100” by Fairmount Chemical, or in micronized form as an aqueous dispersion under the trade name “Tinosorb M” by Ciba Specialty Chemicals;
anthranilic derivatives, including the following: Menthyl anthranilate sold under the trade name “Neo Heliopan MA” by Haarmann and Reimer;
imidazoline derivatives, including the following: Ethylhexyldimethoxybenzylidenedioxoimidazoline propionate;
benzalmalonate derivatives, including the following: Polyorganosiloxanes, containing benzalmalonate functions, sold under the trade name “Parsol SLX” by Hoffmann LaRoche;
   and mixtures thereof.

The organic photoprotective agents that are more particularly preferred are chosen from the following compounds: Ethylhexyl salicylate, Ethylhexyl methoxycinnamate, Octocrylene, Phenylbenzimidazolesulfonic acid, Benzophenone-3, Benzophenone-4, Benzophenone-5, 4-Methylbenzylidene-camphor, Terephthalylidenedicamphorsulfonic acid, Disodium phenyldibenzimidazoletetrasulfonate, 2,4,6-Tris(diisobutyl 4′-aminobenzalmalonate)-s-triazine, Anisotriazine, Ethylhexyltriazone, Diethylhexylbutamidotriazone, Methylenebis(benzotriazolyl)tetramethylbutylphenol, Drometrizole trisiloxane, and mixtures thereof.

The mineral photoprotective agents may be chosen from pigments or even nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminium stearate. Such coated or uncoated metal oxide nanopigments are described in particular in patent applications EP-A-518 772 and EP-A-518 773. Examples that may be mentioned include the hydrophilic nanotitanium sold under the name Mirasun TIW60 by the company Rhodia, and the lipophilic nanotitanium sold under the name MT100T by the company Tayca or UV Titan M 160 by the company Kemira.

When they are present in the compositions according to the invention, the UV-screening agents are generally in proportions ranging from 0.1% to 20% by weight relative to the total weight of the composition, and preferably ranging from 0.2% to 15% by weight relative to the total weight of the composition.

The compositions according to the invention may be in any form including the form of aqueous gels or of more or less fluid emulsions constituting milks or creams. These compositions are prepared according to the usual methods.

The compositions of the invention may be used especially as care products, medicated products, protective products, cleansing (rinse-out or leave-in) products, makeup-removing products and/or makeup products for keratin materials (skin, hair, scalp, eyelashes, eyebrows, nails or mucous membranes), for example as protective, medicated or care creams for the face, the hands or the body, as protective or care body milks or as care gels or mousses for the skin and/or mucous membranes (lips).

Moreover, the compositions of the invention containing sunscreens may thus also be used as antisun products (protection against sunlight and/or UV from tanning machines), especially for the skin and/or the hair.

The compositions may also be used as makeup products, especially for making up the skin, the eyebrows, the eyelashes and the lips, such as face creams, foundations, mascaras or lipsticks. Such products generally contain pigments.

Thus, a subject of the invention is the cosmetic use of a cosmetic composition as defined above, as a care product, a makeup product, an antisun product or a cleansing and/or makeup-removing product.

Another subject of the invention is a cosmetic process (non-therapeutic) for treating a keratin material such as the skin, the scalp, the hair, the eyelashes, the eyebrows, the nails or mucous membranes, wherein a cosmetic composition as defined above is applied to the keratin material.

The invention is illustrated in greater detail in the examples that follow.

### I. EXAMPLES OF SYNTHESIS OF POLYMERS

### Example I/1

### Synthesis by Grafting an AMPS-based Water-soluble Polymer and an (Oxyethylene)₆ b (Oxypropylene)₄₀ Butyl Ether Diblock Polymer

3.78 grams of a copolymer of acrylic acid (20 mol %) and of acrylamido-2-methylpropanesulfonic acid (80 mol %) with a mean molar mass of 500 000 g/mol are dissolved in 715 ml of deionized water with stirring at 20° C. for 12 hours, in a 1 litre reactor equipped with a condenser. The pH of the reaction medium is then adjusted to 8 using 1M sodium hydroxide solution.

Separately, 1.60 grams of (oxyethylene)₆ b (oxypropylene)₄₀ butyl ether monoamino diblock copolymer are dissolved in 100 ml of deionized water with stirring at 5° C. for 30 minutes. The solution obtained is added dropwise to the reaction medium obtained in the reactor, with vigorous stirring.

Next, 1.84 grams of 1-(3-dimethylaminopropyl)-3-ethylcarbodiimide hydrochloride are dissolved in 50 ml of deionized water with stirring at 20° C. for 15 minutes. The solution obtained is added dropwise, with vigorous stirring, to the above reaction medium, which is then heated at 60° C. for 6 hours.

The reaction medium is cooled to 20° C. and then concentrated and precipitated from acetone. The polymer in solid form is recovered by filtration and washed with excess acetone. The powder is dried and ground under vacuum at 35° C.

5.5 g of a copolymer of acrylic acid (20 mol %) and of acrylamido-2-methylpropanesulfonic acid (80 mol %) with a mean molar mass of 500 000 g/mol grafted with 3 mol % of (oxyethylene)₆ b (oxypropylene)₄₀ butyl ether (OE)₆(OP)₃₉ diblock copolymer are obtained (94% yield).

Example I/2

### Synthesis by Grafting an AMPS-based Water-soluble Polymer and an (Oxyethylene)₂₀ b (Oxypropylene)₆₉ b (Oxyethylene)₂₀ Triblock Polymer (Pluronic P123)

3.78 grams of a copolymer of acrylic acid (20 mol %) and of acrylamido-2-methylpropanesulfonic acid (80 mol %) with a mean molar mass of 500 000 g/mol are dissolved in 1385 ml of deionized water with stirring at 20° C. for 12 hours, in a 1 litre reactor equipped with a condenser. The pH of the reaction medium is then adjusted to 8 using 1M sodium hydroxide solution.

4.83 grams of (oxyethylene)₂₀ b (oxypropylene)₆₉ b (oxyethylene)₂₀ block copolymer (Pluronic P123), monoaminated beforehand, are dissolved in 100 ml of deionized water with stirring at 5° C. for 30 minutes. The solution obtained is added dropwise to the reaction medium obtained in the reactor, with vigorous stirring.

2.41 grams of 1-(3-dimethylaminopropyl)-3-ethylcarbodimide hydrochloride are dissolved in 50 ml of deionized water with stirring at 20° C. for 15 minutes. The solution obtained is added dropwise, with vigorous stirring, to the above reaction medium, which is then heated at 60° C. for 6 hours.

The reaction medium is cooled to 20° C. and then concentrated and precipitated from acetone. The polymer in solid form is recovered by filtration and washed with excess acetone. The powder is ground and dried under vacuum at 35° C.

8.5 g of a copolymer of acrylic acid (20 mol %) and of acrylamido-2-methylpropanesulfonic acid (80 mol %) with a mean molar mass of 500 000 g/mol grafted with 4 mol % of (oxyethylene)₂₀ b (oxypropylene)₆₉ b (oxyethylene)₂₀ triblock copolymer are obtained (94% yield).

### Example I/3

### Synthesis by Copolymerization of an AMPS-based Water-soluble Polymer and of an (Oxyethylene)₁₀₆ b (Oxypropylene)₆₉ b (Oxyethylene)₁₀₆ Triblock Polymer (Pluronic F127)

300 grams of tert-butanol are introduced into a 2 litre reactor equipped with a stirrer, a condenser, a thermometer, a thermostatically regulated bath, a reagent dropping funnel and a gas inlet to control the atmosphere above the reaction medium. The reaction medium is placed under a nitrogen atmosphere, after bubbling nitrogen through for 30 minutes. 60 grams of acrylamido-2-methylpropanesulfonic acid (AMPS) are introduced with stirring and under a stream of nitrogen; the pH of the reaction medium is then equal to 1. Ammonia gas is introduced over the reaction medium until its pH is between 7 and 8. Next, 60 grams of (oxyethylene)₁₀₆ b (oxypropylene)₆₉ b (oxyethylene)₁₀₆ triblock copolymer methacrylate are introduced into the reaction medium, which is then stirred for 1 hour, the pH being measured continuously to check that its value is between 7 and 8. The atmosphere above the reaction medium is again saturated with nitrogen in order to reduce the oxygen content in the liquid phase to a level below 1 ppm. 1 gram of azobisisobutyronitrile (AIBN) is then introduced into the reaction medium under nitrogen, and the temperature is adjusted to 60° C. Once the temperature reaches 60° C., the polymerization reaction starts. After about 30 minutes, the temperature is adjusted to the boiling point of the tert-butanol; the reaction medium is then refluxed with stirring for 2 hours.

The reaction medium then becomes a viscous suspension of polymer in tert-butanol, and the polymer is recovered by simple filtration of the tert-butanol, followed by drying under vacuum.

The copolymer obtained is composed of 50% by mass of AMPS (ammonium salt) and 50% of (oxyethylene)₁₀₆ b (oxypropylene)₆₉ b (oxyethylene)₁₀₆ triblock copolymer methacrylate, which corresponds to a molar proportion of 1.65% of triblock copolymer in the final copolymer.

### Example I/4

### Synthesis by Copolymerization of an AMPS-based Water-soluble Polymer and of (Oxyethylene)₁₀ b (Oxypropylene)₃₀ Stearyl Ether Diblock Polymer

300 grams of tert-butanol are introduced into a 2 litre reactor equipped with a stirrer, a condenser, a thermometer, a thermostatically regulated bath, a reagent dropping funnel and a gas inlet to control the atmosphere above the reaction medium. The reaction medium is placed under a nitrogen atmosphere, after bubbling nitrogen through for 30 minutes. 60 grams of acrylamido-2-methylpropanesulfonic acid (AMPS) are introduced with stirring and under a stream of nitrogen, the pH of the reaction medium then being equal to 1. Ammonia gas is introduced over the reaction medium until its pH is between 7 and 8. Next, 60 grams of (oxyethylene)₁₀ b (oxypropylene)₃₀ stearyl ether diblock polymer are introduced into the reaction medium which is then stirred for 1 hour, the pH being measured continuously to check that its value is between 7 and 8. The atmosphere above the reaction medium is again saturated with nitrogen to reduce the oxygen content in the liquid phase to a content below 1 ppm. Next, 1 gram of azobisisobutyronitrile (AIBN) is introduced into the reaction medium under nitrogen, and the temperature is adjusted to 60° C. Once the temperature reaches 60° C., the polymerization reaction starts. After about 30 minutes, the temperature is adjusted to the boiling point of the tert-butanol; the reaction medium is then refluxed with stirring for 2 hours.

The reaction medium then becomes a viscous suspension of polymer in the tert-butanol, and the polymer is recovered by simple filtration of the tert-butanol, followed by drying under vacuum.

The copolymer obtained is composed of 50% by mass of AMPS (ammonium salt) and 50% of (oxyethylene)₁₀ b (oxypropylene)₃₀ stearyl ether diblock copolymer methacrylate, which corresponds to a molar proportion of 8.5% of diblock copolymer in the final copolymer.

### II. COMPOSITION EXAMPLES

The amounts indicated are in percentages by weight, unless otherwise mentioned.

### Example II/1 According to the Invention

### Moisturizing Gel Comprising the Polymer of Example I/1

| | |
|---|---|
| Polymer of Example I/1 | 2 g |
| Glycerol | 3 g |
| Preserving agent | 0.2 g |
| Demineralized water | 94.8 g |

The heat-gelling power of this composition was demonstrated via rheological measurements (Haake RS150 rheometer, cone/plate 35 mm/2°), at a shear rate of 10 s-¹:
viscosity at 20° C.: 0.02 Pa.s
viscosity at 32° C.: 50 Pa.s
gel point: 25° C.

### Comparative Example II/2

### Moisturizing Gel Comprising a Copolymer of Acrylic Acid (20 Mol %) and of AMPS (80 mol %) Bearing 3 Mol % of (Oxyethylene)₆ (Oxypropylene)₄₀ Butyl Ether Random Copolymer Side Chains

| | |
|---|---|
| Copolymer of acrylic acid (20 mol %) and | 2 g |
| of AMPS (80% mol %) bearing 3 mol % of | |
| (oxyethylene)₆(oxypropylene)₄₀ butyl ether | |
| random copolymer side chains | |
| Glycerol | 3 g |
| Preserving agent | 0.2 g |
| Demineralized water | 94.8 g |

The heat-gelling power of this composition was demonstrated via rheological measurements (Haake RS150 rheometer, cone/plate 35 mm/2°), at a shear rate of 10 s-¹:
viscosity at 20° C.: 0.015 Pa.s
viscosity at 32° C.: 1 Pa.s
gel point: 28° C.

This composition has a gel point close to that of the composition of Example II/1 according to the invention, but the amplitude of the heat-gelling effect between 20° C. and 32° C. is much smaller. The fluid/gel transition on application is less pronounced and leads to less pronounced sensory effects.

### Example II/3 According to the Invention

### Body Milk

Aqueous phase:

| | |
|---|---|
| Polymer of Example I/2 | 0.4 g |
| Preserving agent | 0.2 g |
| Demineralized water | 84.4 g |

Oily phase:

| | |
|---|---|
| Parleam oil | 9 g |
| Cyclohexadimethylsiloxane | 6 g |

The aqueous phase is prepared by dissolving the polymer of Example I/2 at room temperature in demineralized water containing the preserving agent, with stirring for 2 hours. The oily phase is then introduced slowly into the aqueous phase with stirring using a Moritz blender at a speed of 4000 rpm for 20 minutes.

The polymer of Example I/2 by itself allows all of the oily phase to be emulsified. The composition obtained is an attractive fluid emulsion that may be used as abody milk.

The above written description of the invention provides a manner and process of making and using it such that any person skilled in this art is enabled to make and use the same, this enablement being provided in particular for the subject matter of the appended claims, which make up a part of the original description and including an aqueous composition for topical application, containing a physiologically acceptable medium and at least one water-soluble polymer consisting of a water-soluble skeleton based on 2-acrylamido-2-methyl-propanesulfonic acid and of side chains comprising 1) at least one polyoxyethylene block and 2) at least one polyoxypropylene block and/or at least one one polyoxybutylene block.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

As used herein, where a certain polymer is noted as being “obtained from” or “comprising”, etc. one or more monomers (or units) this description is of the finished polymer material itself and the repeating units therein that make up, in whole or part, this finished product. One of ordinary skill in the art understands that, speaking precisely, a polymer does not include individual, unreacted “monomers,” but instead is made up of repeating units derived from reacted monomers.

1. An aqueous composition for topical application, comprising water and at least one water-soluble polymer, said water-soluble polymer comprising a water-soluble 2-acrylamido-2-methylpropanesulfonic acid skeleton and at least one side chain, said side chain comprising 1) at least one polyoxyethylene block and 2) at least one block selected from the group consisting of a polyoxypropylene block and a polyoxybutylene block.

2. The composition according to claim 1, wherein the water-soluble polymer is obtained from:
(A) 70 mol % to 99.5 mol % of 2-acrylamido-2-methylpropanesulfonic acid units of formula (I): 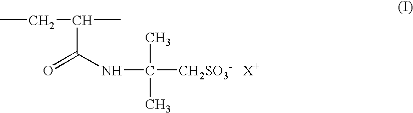
in which X⁺ is a proton, an alkali metal cation, an alkaline-earth metal cation or an ammonium ion;
(B) 0.5 mol % to 30 mol % of units of formula (II) below: 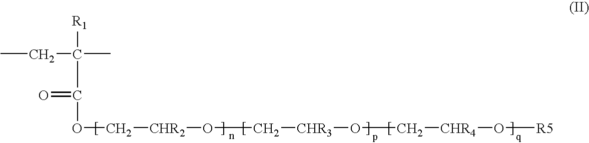
in which:
n, p and q, independently of each other, denote an integer and range from 0 to 200;
R₁ denotes a hydrogen atom or a linear or branched alkyl radical containing from 1 to 6 carbon atoms;
R₂, R₃ and R₄ denote a hydrogen atom or an alkyl radical containing 1 or 2 carbon atoms, at least one of the radicals R₂, R₃ or R₄ being a hydrogen atom, and at least one of the radicals R₂, R₃ or R₄ being an alkyl radical containing one or two carbon atoms;
R₅ denotes a hydrogen atom or a linear, cyclic or branched alkyl radical containing from 1 to 18 carbon atoms; and
(C) 0 to 30 mol % of water-soluble or hydrophobic units bearing, before polymerization, at least one unsaturated function.

3. The composition according to claim 2, wherein the unit of formula (II) is chosen from those in which:
one of the radicals R₂ or R₃ is a hydrogen atom and the other is an alkyl radical containing one or two carbon atoms, and the integer q is equal to 0; or alternatively
R₂ is a hydrogen atom, R₃ is a methyl radical and R₄ and R₅ are hydrogen atoms; or alternatively
R₂ is a hydrogen atom, R₃ is an ethyl radical and R₄ and R₅ are hydrogen atoms.

4. The composition according to claim 2, wherein the water-soluble unit (C) is present and is obtained from one or more water-soluble monomers selected from the group consisting of the following monomers, which may be in acid form and in salt form:
(meth)acrylic acid;
styrenesulfonic acid;
vinylsulfonic acid and (meth)allylsulfonic acid;
(meth)acrylamide;
vinylphosphonic acid;
N-vinylacetamide and N-methyl-N-vinylacetamide;
N-vinylformamide and N-methyl-N-vinylformamide;
N-vinyllactams comprising a cyclic alkyl group of 4 to 9 carbon atoms;
maleic anhydride;
itaconic acid;
vinyl alcohol;
vinyl ethers of formula CH₂═CHOR in which R is a linear or branched, saturated or unsaturated hydrocarbon-based radical containing from 1 to 6 carbons;
dimethyldiallylammonium chloride;
quaternized dimethylaminoethyl methacrylate;
(meth)acrylamidopropyltrimethylammonium chloride;
methylvinylimidazolium chloride;
2-vinylpyridine and 4-vinylpyridine;
acrylonitrile;
glycidyl (meth)acrylate;
vinyl chloride and vinylidene chloride;
vinyl monomers of formula (A) below: 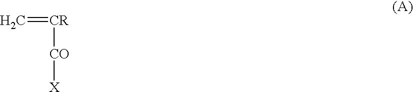
in which:
R is chosen from H, —CH₃, —C₂H₅ and —C₃H₇;
X is chosen from:
alkyl ethers of —OR′ type in which R′ is a linear or branched, saturated or unsaturated hydrocarbon-based radical containing from 1 to 6 carbon atoms, optionally substituted with at least one halogen atom (iodine, bromine, chlorine or fluorine); a sulfonic (—SO₃⁻), sulfate (—SO₄⁻), phosphate (—PO₄H₂); hydroxyl (—OH); primary amine (—NH₂); secondary amine (—NHR₆), tertiary amine (—NR₆R₇) or quaternary amine (—N⁺R₆R₇R₈) group with R₆, R₇ and R₈ being, independently of each other, a linear or branched, saturated or unsaturated hydrocarbon-based radical containing 1 to 6 carbon atoms, with the proviso that the sum of the carbon atoms of R′+R₆+R₇+R₈ does not exceed 6;
—NH₂, —NHR′ and —NR′R″ groups in which R′ and R″ are, independently of each other, linear or branched, saturated or unsaturated hydrocarbon-based radicals containing 1 to 6 carbon atoms, with the proviso that the total number of carbon atoms of R′+R″ does not exceed 6, the said R′ and R″ optionally being substituted with a halogen atom (iodine, bromine, chlorine or fluorine); a hydroxyl (—OH); sulfonic
(—SO₃⁻); sulfate (—SO₄⁻); phosphate (—PO₄H₂); primary amine (—NH₂); secondary amine (—NHR₆), tertiary amine (—NR₆R₇) and/or quaternary amine (—N⁺R₆R₇R₈) group with R₆, R₇ and R₈ being, independently of each other, a linear or branched, saturated or unsaturated hydrocarbon-based radical containing 1 to 6 carbon atoms, with the proviso that the sum of the carbon atoms of R′+R″+R₆+R₇+R₈ does not exceed 6;
and mixtures thereof.

5. The composition according to claim 2, wherein the hydrophobic unit (C) is present and is obtained from one or more hydrophobic monomers selected from the group consisting of the following monomers:
styrene and its derivatives,
vinyl acetate,
vinyl ethers of formula CH₂═CHOR in which R is a linear or branched, saturated or unsaturated hydrocarbon-based radical containing from 1 to 6 carbon atoms;
acrylonitrile,
caprolactone,
vinyl chloride and vinylidene chloride,
silicone derivatives, leading, after polymerization, to silicone polymers,
the hydrophobic vinyl monomers of formula (B) below: 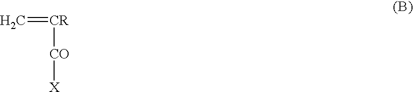
in which:
R is chosen from H, —CH₃, —C₂H₅ and —C₃H₇,
X is chosen from:
alkyl ethers of the type —OR′ in which R′ is a linear or branched, saturated or unsaturated hydrocarbon-based radical containing from 1 to 6 carbon atoms;
groups —NH₂, —NHR′ and —NR′R″ in which R′ and R″ are, independently of each other, linear or branched, saturated or unsaturated hydrocarbon-based radicals containing from 1 to 6 carbon atoms, with the proviso that the total number of carbon atoms in R′+R″ does not exceed 6;
and mixtures thereof.

6. The composition according to claim 2, wherein the water-soluble skeleton and/or the water-soluble unit (C) are partially or totally neutralized with a mineral base or an organic base.

7. The composition according to claim 1, wherein the water-soluble skeleton is crosslinked with a crosslinking agent chosen from methylenebisacrylamide, allyl methacrylate and trimethylolpropane triacrylate.

8. The composition according to claim 1, wherein the water-soluble skeleton has a molar mass of between 10 000 g/mol and 10 000 000 g/mol.

9. The composition according to claim 1, wherein the polymer is obtained by a reaction for grafting polyether chains having at least one reactive end onto water-soluble macromolecules bearing complementary reactive sites, or by copolymerization of a polyether macromonomer and at least one water-soluble monomer.

10. The composition according to claim 1, wherein the polymer is present in an amount ranging from 0.01% to 20% by weight relative to the total weight of the composition.

11. The composition according to claim 1, wherein it contains an amount of water ranging from 20% to 99.9% by weight relative to the total weight of the composition.

12. The composition according to claim 1, further comprising
an oily phase.

13. The composition according to claim 1, wherein it is a cosmetic or dermatological composition.

14. The composition according to claim 1, wherein it is a care product, a medicated product, a protective product, a cleansing product, a makeup-removing product and/or a makeup product for keratin materials.

15. A process comprising applying the composition of claim 1 to keratin material.

16. The process of claim 15, wherein said composition is a care product, a makeup product, an antisun product or a cleansing and/or makeup-removing product.

17. The process of claim 15, wherein said composition is applied to the skin.

18. The process of claim 16, wherein said composition is applied to the skin.

19. A process comprising applying the composition of claim 1 to keratin material, wherein said composition is a cosmetic composition.

20. The process of claim 19, wherein said composition is applied to the skin.
